(19) United States
(12) Patent Application Publication
(10) Pub. No.: US 2014/0112978 A1
(43) Pub. Date: Apr. 24, 2014

(54) IBUPROFEN-BASED COMPOUND, PREPARATION METHOD, USE, AND FORMULATION OF THE SAME

(71) Applicants: Qing Su, Daqing, Heilongjiang (CN); Guoqing Leng, Daqing, Heilongjiang (CN); Zhiguang Song, Changchun, Jilin (CN); Wenge Hou, Changchun, Jilin (CN); Xi Chen, Changchun, Jilin (CN)

(72) Inventors: Qing Su, Daqing, Heilongjiang (CN); Guoqing Leng, Daqing, Heilongjiang (CN); Zhiguang Song, Changchun, Jilin (CN); Wenge Hou, Changchun, Jilin (CN); Xi Chen, Changchun, Jilin (CN)

(73) Assignee: Hellongjoamg Baoqinglong Biotechnology Co. Ltd., Daqing, Heilongjiang (VN)

(21) Appl. No.: 14/124,390

(22) PCT Filed: May 23, 2012
(86) PCT No.: PCT/CN2012/075927

§ 371 Date: Dec. 6, 2013

(30) Foreign Application Priority Data
Jul. 21, 2011 (CN) 201110205563.0

(51) Int. Cl. C07C 69/612

(52) U.S. Cl. CPC C07C 69/612
USPC 424/450; 560/103; 514/533

(57) ABSTRACT

Disclosed are compounds based on ibuprofen, their preparation methods, uses and pharmaceutical preparation. The compounds have structures shown as formula (1), wherein, m, n are integers and fulfill the requirements of 0≦n≦6, 0≦m≦6, respectively. The preparation methods for the compounds based on ibuprofen are as follows: contacting and reacting 2-(4-isobutyl-phenyl) propionic acid to have contact reaction with a solution of an organic acid ester in the presence of a catalyst under substitution reaction conditions The present compounds can be used to prepare nonsteroidal anti-inflammatory drugs. The preparation can be preparation of fat emulsion, liposome, and dried emulsion and so on.

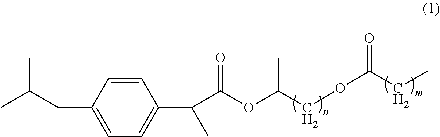

### FIELD OF THE INVENTION

The present invention relates to an ibuprofen-based compound, a method for preparation of the compound, and use of the compound in preparation of non-steroidal anti-inflammatory drugs (NSAIDs).

### BACKGROUND OF THE INVENTION

Ibuprofen, with the chemical name as 2-(4-isobutyl phenyl) propionic acid, has analgesic, anti-inflammatory, and anti-pyretic effects, and is a non-steroidal anti-inflammatory drug (NSAID) that is used the most widely in the world at present. However, owing to the fact that ibuprofen has stronger inhibiting effect for COX-1 (cyclooxygenase-1) than for COX-2, ibuprofen may cause severe side effects to the gastrointestinal tract (including gastrointestinal bleeding, perforation, and pylorochesis, etc.) up to 20%˜50% probability, and such hazard may be fatal to some patients. FDA reports have indicated: NSAIDs may induce upper gastrointestinal hemorrhage, massive haemorrhage or perforation. The probability of occurrence is 1% among patients treated with NSAIDs for 3˜6 months and 2%˜4% among patients treated with NSAIDs for 1 year; in addition, the probability will increase continuously as the treatment time increases (Chinese Journal of New Drugs 2009, 18(6):497-501).

Conventional NSAIDs inhibit both COX-1 and COX-2, and have side effects to gastrointestinal tract and kidneys. COX-2 selective inhibitors can avoid or minimize the side effects to gastrointestinal tract while they exert anti-inflammatory and analgesic effects. After the Rofecoxib Event, the pharmaceutical industry re-examined the research direction of NSAIDs selective COXs. In recent years, pharmaceutical researchers paid their attention to the research for structural optimization of ibuprofen.

In their research findings, GUO Changbin et al believed: ibuprofen lacks of structural segments that can occupy the side pockets of COX-2; therefore, ibuprofen has no selectivity to the two types of isoenzymes. In view of that, they designed a target compound that could substitute benzoylamino at the third position of the benzene ring of ibuprofen, so as to occupy the side pockets of COX-2 and enhance conjugation with COX-2 (ACTA CHIMICA SINICA 2005, 63(9):841-848).

To reduce the side effect of ibuprofen to the gastrointestinal tract and improve the anti-inflammatory activity of ibuprofen, SONG Ni et al chose typical monosaccharide and disaccharide and controlled the hydroxyl, 1-amino and 2-amino in the sugar ring to have acylation reaction with the carboxyl in ibuprofen molecule, to couple the ibuprofen molecule with the sugar ring part and thereby produce sugar derivatives of ibuprofen (Acta Pharmaceutica Sinica 2004, 39(2):105-109).

ZHAO Xiuli et al from Shenyang Pharmaceutical University invented an eugenol ibuprofen ester produced from ibuprofen, by chloroformylation reaction to form anhydride, esterification reaction in an organic solvent, and recrystallization (CN1597656A).

HU Aixi et al from Hunan University dissolved ibuprofen chloride in tetrahydrofuran and added 4-ethoxyl-2-aryl-morpholin-tetrahydrofuran solution in droplets to prepare ibuprofen-2-aryl-morpholin-ethyl ester; dissolved the ibuprofen-2-aryl-morpholin-ethyl ester in anhydrous ether or alcohol and fed dry HCl gas or appropriate acid (HY) to react, to obtain a salt of ibuprofen-2-aryl-morpholin-ethyl ester (CN101812033A).

SUN Lilin et al from Anhei Normal University bonded NASID ibuprofen to double-bond 2-Hydroxyethyl methacrylate (HEMA) via covalent bonds to produce an ibuprofen-containing monomer, and then synthetized an ibuprofen-containing polymeric drug by self-polymerization or copolymerization. The author expected to attain controlled-release of the drug by hydrolysis or enzymolysis of the chemical bonds, so as to obtain better pharmacological performance and avoid some side effects (Journal of Functional Polymers 2004, 17(1):97-101).

SHANG Rui et al from University of Science and Technology of China, on the basis of ibuprofen synthesis, synthesized ketoprofen, suprofen, and fenoprofen from halogeno-benzene derivatives and cyanoacetate derivatives, in order to obtain a NASID that is safe and reliable clinically (CN102010323A).

The prior art has the following drawbacks:

1. Modify the benzene ring structure of ibuprofen to obtain a COX-2 selective inhibitor. Though the obtained compound has enhanced conjugation with COX-2, the inhibiting effect of the compound is reduced both to COX-1 and COX-2 after structural modification, and the medicinal effect is degraded. It is conjectured that the introduced group brings severe change to the structure of ibuprofen and results in changed pharmacological activity.

2. The medicinal effect is also degraded for a complex compound of ibuprofen obtained by ibuprofen conjugation and esterification since the structure of ibuprofen is changed severely. The reason may be that the complex compound of ibuprofen changes the pharmacological action in the metabolic process in human body and results in degraded anti-inflammatory or analgesic effect.

3. The drug toxicity of ketoprofen, suprofen, and fenoprofen synthesized from halogeno-benzene derivatives and cyanoacetate derivatives: though the pharmacological action in an aspect (e.g., analgesic or anti-inflammatory effect) is enhanced, is changed, increasing adverse effects (e.g., gastrointestinal irritation).

4. For the mixed injection of ibuprofen and arginine prepared with arginine as the booster solvent (U.S. Pat. No. 6,727,286B2), a large volume of normal saline is required to dilute the injection to avoid hemolysis in the injection project; in addition, the pH of the diluting normal saline has to be controlled strictly; otherwise the active component in the drug (i.e., ibuprofen) will precipitate or be degraded. The mixed injection of ibuprofen and arginine may result in decreased drug stability under temperature effect; therefore, the sterilization conditions and effect of the injection are limited.

Therefore, it is a demand to develop a drug that doesn't decrease the favorable medicinal effect of ibuprofen and can effectively inhibit the side effects of ibuprofen, and produce the drug into ibuprofen injection that has stable chemical properties without destroying active components, and can be used for intravenous injection.

### SUMMARY OF THE INVENTION

The object of the present invention is to provide a new ibuprofen-based compound, especially ibuprofen-1-acetoxy ethyl ester, or (R)-(−)-ibuprofen-1-acetoxy ethyl ester, or (S)-(+)-ibuprofen-1-acetoxy ethyl ester, to overcome the drawbacks in the prior art.

To attain the object described above, the present invention provides an ibuprofen-based compound, which has the structure represented by structural formula (1):

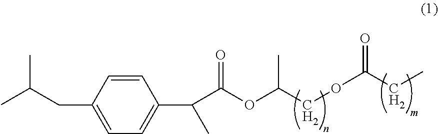

Wherein, 0≦n≦6, 0≦m≦6, and m, n are integers.

The present invention further provides a method for preparation of ibuprofen-based compound, comprising: controlling 2-(4-isobutyl-phenyl) propionic acid to contact with an organic acid ester solution represented by structural formula (5), under substitution reaction conditions, with the existence of a catalyst;

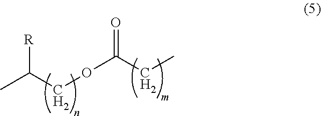

Wherein, 0≦n≦6, 0≦m≦6, m and n are integers, and R is a haloid element or

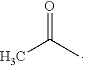

The present invention further provides a use of the above compound in preparation of NSAIDs. The present further provides a formulation containing the above compound.

The ibuprofen ester-based compound provided in the present invention has high liposolubility, and can be produced into a stable formulation for intravenous injection, such as nano-size emulsion or liposome injection, etc. The intravenous injection has high targeting effect, can effectively accumulate ibuprofen drug at the inflamed part and selectively inhibit COX-2. Pharmacokinetic tests have proved that the intravenous injection can take effect quickly and last for long action time. In addition, after sterilization at high temperature, the intravenous injection emulsion has average particle size within 160˜190 nm range, with maximum particle size not greater than 330 nm; therefore, it can be used directly for intravenous injection without diluting with normal saline, and is especially suitable for patients who suffer pain before and after operation.

The ibuprofen ester-based compound provided in the present invention can be used not only for preparation of formulations for intravenous injection but also for preparation of oral micro-emulsion formulations. White rat oral administration tests have proved that the micro-emulsion formulation leaves little drug residue in oral cavity and esophagus, and nearly no impairment to gastric mucosa and intestinal tract is seen. Pharmacokinetic tests have proved that the oral emulsion improves the bioavailability of ibuprofen drug and prolong the action time of ibuprofen drug.

Other characteristics and advantages of the present invention will be further detailed in the embodiments hereunder.

### BRIEF DESCRIPTION OF THE DRAWINGS

The accompanying drawings are provided here to facilitate further understanding on the present invention, and are a part of this specification. They are used together with the following embodiments to explain the present invention, but shall not be comprehended as constituting any limitation to the present invention. Among the drawings:

FIG. 1 is an infrared spectrogram of the target compound in Example 1.

FIG. 2 is a nuclear magnetic resonance spectrogram of the target compound in Example 1.

FIG. 3 is a mass spectrogram of the target compound in Example 1.

FIG. 4 is an emulsion particle size distribution diagram of the Example 12 after sterilization.

FIG. 5 is an emulsion particle size distribution diagram of the Example 13 after sterilization.

FIG. 6 is an emulsion particle size distribution diagram of the Example 14 after sterilization.

FIG. 7 is an emulsion particle size distribution diagram of the Example 15 after sterilization.

FIG. 8 is an emulsion particle size distribution diagram of the Example 16 after sterilization.

FIG. 9 shows the drug-time curve of the mid-chain/long-chain lipid emulsion of ibuprofen-1-acetoxy ethyl ester in test group 1 in Example 22 after intravenous injection.

FIG. 10 shows the drug-time curve of the mid-chain/long-chain lipid emulsion of ibuprofen-1-acetoxy ethyl ester in test group 2 in Example 22 after oral dosing.

FIG. 11 shows the mean drug-time curve of mid-chain/long-chain lipid emulsion of ibuprofen-1-acetoxy ethyl ester in test group 2 in Example 22 after oral administration.

FIG. 12 shows the drug-time curve of the ibuprofen injection in comparative group 1 in Comparative Example 1 after intravenous injection.

FIG. 13 shows the mean drug-time curves of the ibuprofen injection in comparative group 1 in Comparative Example 1 after intravenous injection and the mid-chain/long-chain lipid emulsion of ibuprofen-1-acetoxy ethyl ester in test group 1 in Example 22 after intravenous injection.

FIG. 14 shows the drug-time curves of the ibuprofen injection in comparative group 1 in Comparative Example 1 and the mid-chain/long-chain lipid emulsion of ibuprofen-1-acetoxy ethyl ester in test group 1 in Example 22 within 1 h after intravenous injection.

### DETAILED DESCRIPTION OF THE EMBODIMENTS

The present invention provides an ibuprofen-based compound, which has the structure represented by structural formula (1):

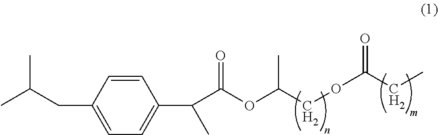

Where, 0≦n≦6, 0≦m≦6, and m, n are integers.

For the compound provided in the present invention, the value of m can be 0, 1, 2, 3, 4, 5, or 6, and the value of n can be 0, 1, 2, 3, 4, 5, or 6, and the structure of the compound can be a combination of the values of m and n. For example, the compound can be one or more selected from the group consisting of ibuprofen-1-acetoxy (or propionyloxy, or butyryloxy, or valeryloxy, or hexenoyloxy, or enanthyloxy, or octanoyloxy) ethyl ester, ibuprofen-1-acetoxy (or propionyloxy, or butyryloxy, or valeryloxy, or hexenoyloxy, or enanthyloxy, or octanoyloxy) propyl ester, ibuprofen-1-acetoxy (or propionyloxy, or butyryloxy, or valeryloxy, or hexenoyloxy, or enanthyloxy, or octanoyloxy) butyl ester, ibuprofen-1-acetoxy (or propionyloxy, or butyryloxy, or valeryloxy, or hexenoyloxy, or enanthyloxy, or octanoyloxy) amyl ester, ibuprofen-1-acetoxy (or propionyloxy, or butyryloxy, or valeryloxy, or hexenoyloxy, or enanthyloxy, or octanoyloxy) hexyl ester, ibuprofen-1-acetoxy (or propionyloxy, or butyryloxy, or valeryloxy, or hexenoyloxy, or enanthyloxy, or octanoyloxy) heptyl ester, and ibuprofen-1-acetoxy (or propionyloxy, or butyryloxy, or valeryloxy, or hexenoyloxy, or enanthyloxy, or octanoyloxy) octyl ester.

Preferably, the compound has the structure represented by structural formula (2),

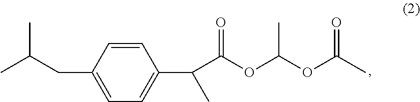 [SMILES: CC(=O)OC(C)OC(=O)C(C)c1ccc(CC(C)C)cc1]

i.e., ibuprofen-1-acetoxy ethyl ester, with molecular formula as C₁₇H₂₄O₄.

In a preferred embodiment, the compound is a levorotatory chiral enantiomer of ibuprofen-1-acetoxy ethyl ester, i.e., (R)-(−)-ibuprofen-1-acetoxy ethyl ester, which has the structure represented by structural formula (3),

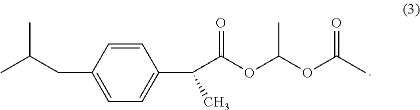 [SMILES: CC(=O)OC(C)OC(=O)[C@H](C)c1ccc(CC(C)C)cc1]

In another preferred embodiment, the compound is a dextrorotatory chiral enantiomer of ibuprofen-1-acetoxy ethyl ester, i.e., (S)-(+)-ibuprofen-1-acetoxy ethyl ester, which has the structure represented by structural formula (4),

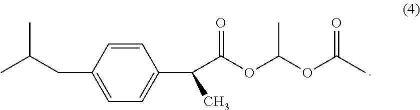 [SMILES: CC(=O)OC(C)OC(=O)[C@@H](C)c1ccc(CC(C)C)cc1]

In the present invention, the optical rotation is measured with a polarimeter measurement method, which is well-known in the art.

The present invention further provides a method for preparation of ibuprofen-based compound, comprising: controlling 2-(4-isobutyl-phenyl) propionic acid to contact with a solution of an organic acid ester represented by structural formula (5), under substitution reaction conditions, with the existence of an catalyst;

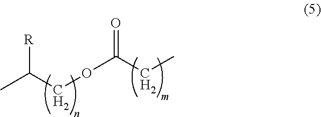

wherein, 0≦n≦6, 0≦m≦6, m and n are integers, and R is a haloid element (e.g., fluorine,
chlorine, bromine, iodine, etc.) or

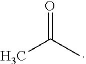

The chemical equation is:

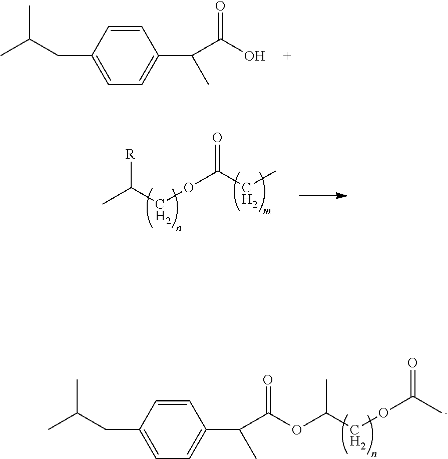

Preferably, m=0, n=0, and the organic acid ester represented by structural formula (5) has the structure represented by structural formula (6),

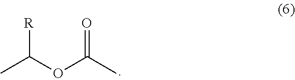

More preferably, R is chlorine, bromine, or

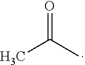

Preferably, the organic acid ester represented by structural formula (5) is one or more selected from the group consisting of 1-ethyl bromoacetate, 1-ethyl chloroacetate, and ethylene diacetate.

Preferably, the 2-(4-isobutyl-phenyl) propionic acid is one or more selected from the group consisting of (R)-2-(4-isobutyl-phenyl) propionic acid and (S)-2-(4-isobutyl-phenyl) propionic acid. The enantiomer can be obtained with chiral solvent extraction and separation method or LC chiral stationary phase separation method, which is well-known in the art.

The substitution reaction conditions in the present invention can be similar to the conditions of nucleophilic substitution reaction between carboxylic acid and halogenated hydrocarbons, and can be conditions that are well known by those skilled in the art. Preferably, the reaction conditions include temperature being of 10-40° C. and reaction time being of 3-10 h.

Preferably, calculated in mole, the ratio of 2-(4-isobutyl-phenyl) propionic acid: organic acid ester represented by structural formula (5) in the solution is 1:1-2, more preferably 1:1.4-1.6. In the present invention, the dosage of the catalyst can be typical dosage. Preferably, the dosage of the catalyst is 10-97% of the weight of 2-(4-isobutyl-phenyl) propionic acid, preferably 12-78%, more preferably 13%-20%.

The catalyst in the present invention can be any ordinary catalyst that is well known in the art which could catalyze the substitution reaction. Preferably, the catalyst can be one or more selected from the group consisting of alkaline catalysts, e.g., one or more of potassium bicarbonate, sodium bicarbonate, sodium carbonate, potassium carbonate, potassium hydroxide, and sodium hydroxide.

The solvent in the solution in the present invention can be any organic solvent that can dissolve the organic acid ester represented by structural formula (5) and doesn't have any adverse effect to the reaction, such as one or more selected from the group consisting of ethanol, ethyl acetate, acetonitrile, 1,4-dioxane, tetrahydrofuran, and acetone.

The dosage of the organic solvent is selected to ensure the concentration of organic acid ester in the organic acid ester solution is preferably 12-72 wt. %, more preferably 15-60 wt. %.

The following chemical equations represent five preferred methods for preparation of the compound, which are:

### First Method:

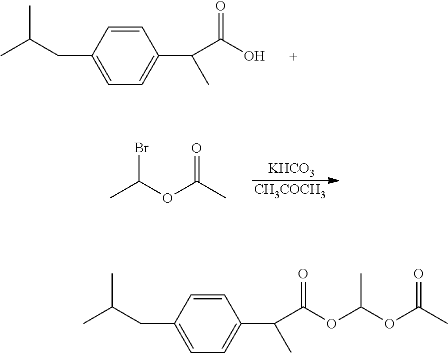

### Second Method:

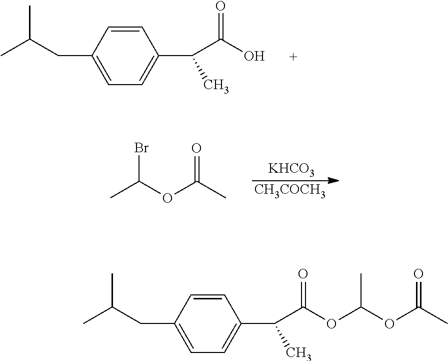

### Third Method:

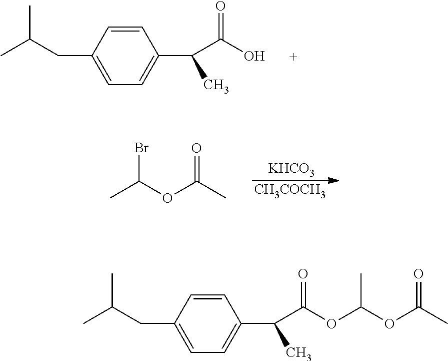

### Fourth Method:

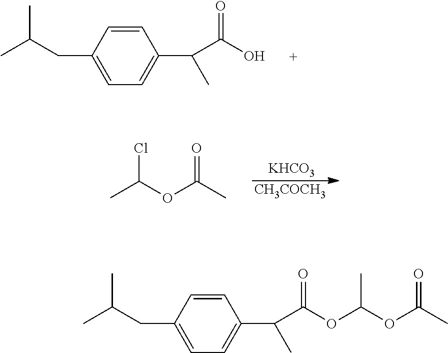

### Fifth Method:

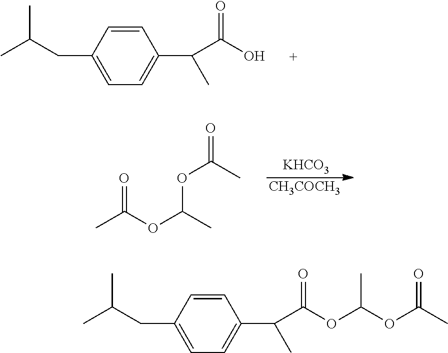

The present invention further provides a use of the above ibuprofen-based compound in preparation of NSAIDs.

The present invention further provides a formulation that contains the above compound, wherein, calculated on the basis of the total weight of the formulation, the content of the ibuprofen-based compound is 1-99 wt. %. Preferably, calculated on the basis of the total weight of the formulation, the content of the ibuprofen-based compound is 25-45 wt. %. More preferably, calculated on the basis of the total weight of the formulation, the content of the ibuprofen-based compound is 28-43 wt. %.

The formulation provided in the present invention can be obtained with method that is well known in the art, and it can be produced into oral emulsion, soft capsule, intravenous injection, etc., or other forms of targeting formulation. Injection, which has better medicinal effect, is preferred.

The injection disclosed in the present invention has high thermal stability, and can be sterilized in water bath under the conditions of 100-126° C. temperature and 8≦F₀<12 or F₀≦12. Viewed from economic efficiency aspect, the sterilization in water bath should be carried out under the conditions of 121° C. temperature and 8≦F₀<12. F₀ is a parameter of heat pressure sterilization, which is well known by those skilled in the art.

COX1 is of structural type, and expresses in many tissues of human body, especially in stomach, kidneys, and platelets, providing state regulation and protection functions; COX2 is of inducible type, mainly related with inflammatory reaction and pain, usually at very low concentration, and is generated in periphery under inflammatory stimulation. The formulation provided in the present invention has high targeting and blood-brain barrier permeability, and can accumulate selectively at inflamed parts (e.g., tumor part, injured blood vessel part, etc.) and operation cut parts; therefore, it can change drug distribution in the body and provide targeting analgesic and anti-inflammatory effect, and can reduce the adverse effects of ibuprofen.

In a preferred embodiment, the compound described in the present invention is dissolved in an oily matrix phase composed of mid-chain fatty acids and long chain fatty acids, and is wrapped by phospholipid membrane to form a nano-size lipid microsphere dispersed system. Lipid microspheres are of a targeting drug carrier, which can accumulate selectively at inflamed tissues and injured blood vessel parts, and thereby changes the drug distribution in the body. Preferably, the formulation is liposome formulation, micro-emulsion formulation, soft capsule, or ointment, etc. More preferably, the formulation is fat emulsion injection, the auxiliary materials of which contain oily matrix phase, lecithin, oleic acid, and glycerol; or, the formulation is frozen dried emulsion injection, the auxiliary materials of which contain oily matrix phase, phosphatidylcholine, oleic acid (or sodium oleate), glycerol and lactose; or, the formulation is liposome injection, the auxiliary materials of which contain phosphatidylcholine, cholesterol, and oleic acid (or sodium oleate). The oily matrix phase is preferably one or more of long-chain fatty acids and mid-chain fatty acids. The obtained injection has stable active component and high re-dissolubility. The mid-chain fatty acids (MCFAs) in the present invention refer to fatty acids with 6-12 carbon atoms in the carbon chain; the long-chain fatty acids (LCFAs) refer to fatty acids with more than 12 carbon atoms in the carbon chain.

The formulation provided in the present invention is applicable to:

1. Relieve rheumatoid pain, acute episode of chronic arthritis, or persistent joint gall.

2. Treat non-articular soft tissue pain and rheumatic pain, and traumatic pain after exercise.

3. Treat post-surgical pain, post-traumatic pain, and post-strain pain.

4. Treat fever incurred by common cold or influenza for adults and children.

The dosage of the compound (calculated in ibuprofen) can be 0.01-20 mg/kg body weight/day; preferably, the dosage in systemic administration (e.g., injection or administration) is 0.25-10 mg/kg body weight/day, and can be administrated in 1-4 cycles. The exact dosage and administrating method depend on the individual difference (e.g., age and state of illness) of the patient.

Hereunder the present invention will be further detailed in some embodiments; however, the examples are provided here only to interpret the preparation method and purpose of the present invention, instead of constituting any limitation to the present invention.

Examples 1-11 are provided to prepared the compounds of the present invention.

### EXAMPLE 1

Add 10.3 g (0.05 mol) ibuprofen and 8 g potassium bicarbonate into a 250 ml three-neck flask, add 110 ml acetone while agitating, add 13.4 g (0.08 mol) 1-ethyl bromoacetate in droplets at room temperature, and maintain the reaction for 5 h while agitating at 25° C.; then, add 200 ml ethyl acetate to dilute the solution, and transfer the reaction liquid into a separatory funnel; wash with 3 wt. % sodium carbonate solution (2×100 ml), and separate to obtain the organic layer; dry with anhydrous sodium sulfate, filter off the drying agent, add active carbon to carry out decolorization with reflux for 20 min., filter off the active carbon, condense the filtrate at normal pressure till no liquid can be distilled off; distil the residue at reduced pressure, and collect 164˜166° C./2 mmHg distillate to obtain 12.6 g colorless liquid, which is the target product ibuprofen-1-acetoxy ethyl ester; in relation to the raw material, the yield ratio of ibuprofen is 86.3%.

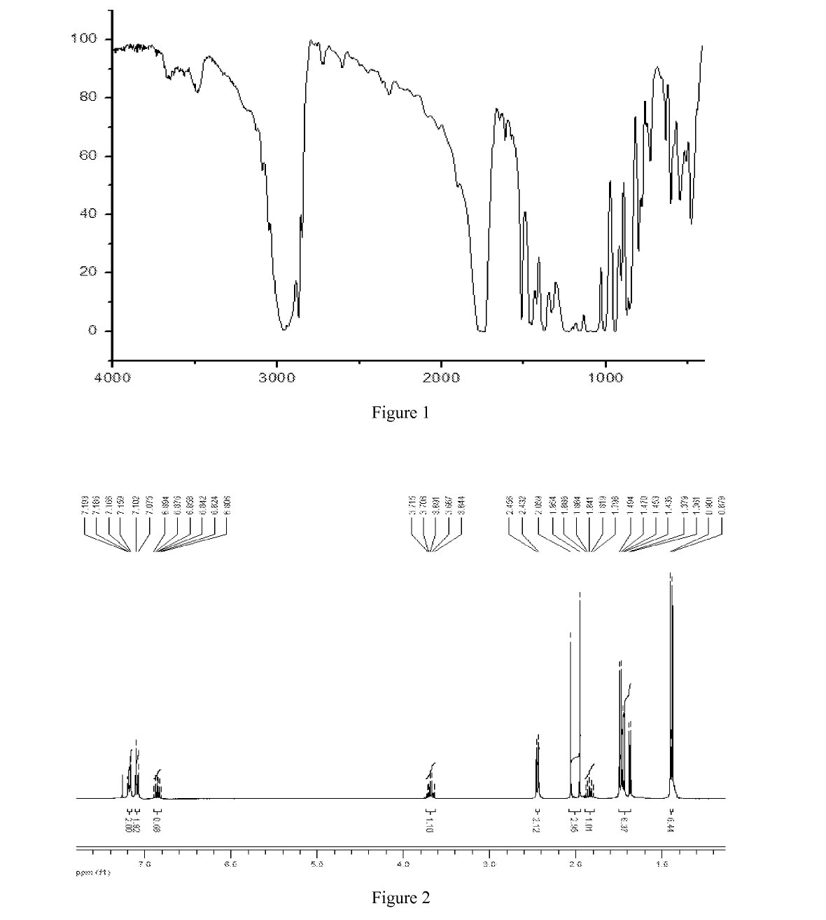

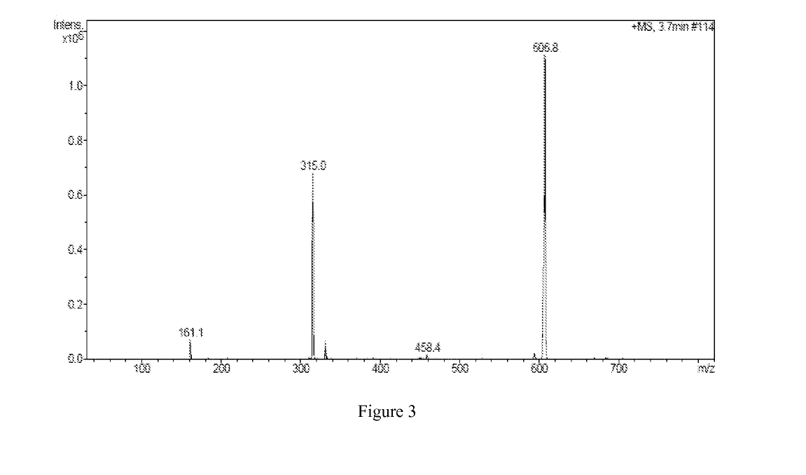

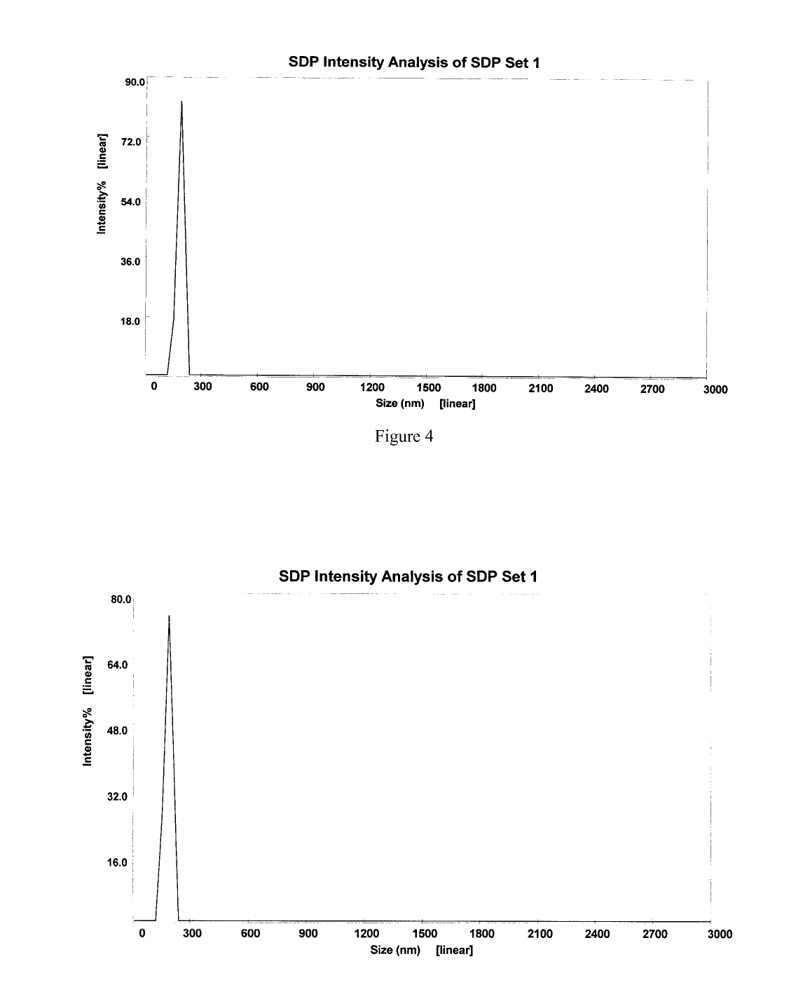

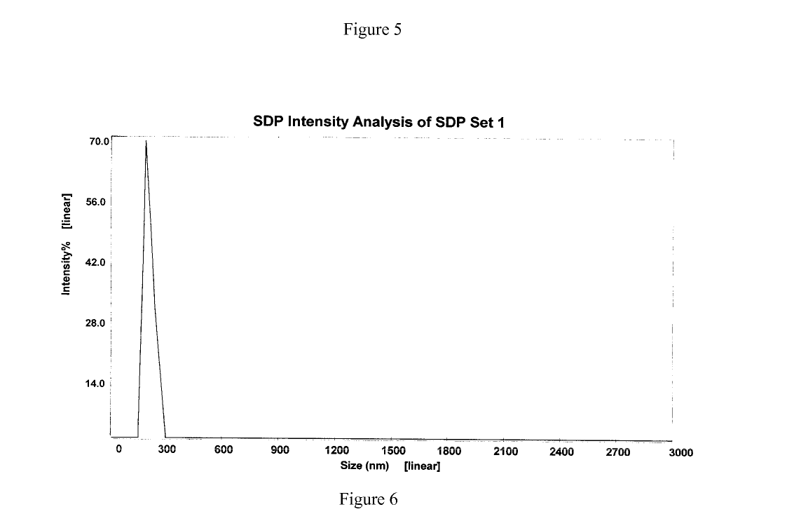

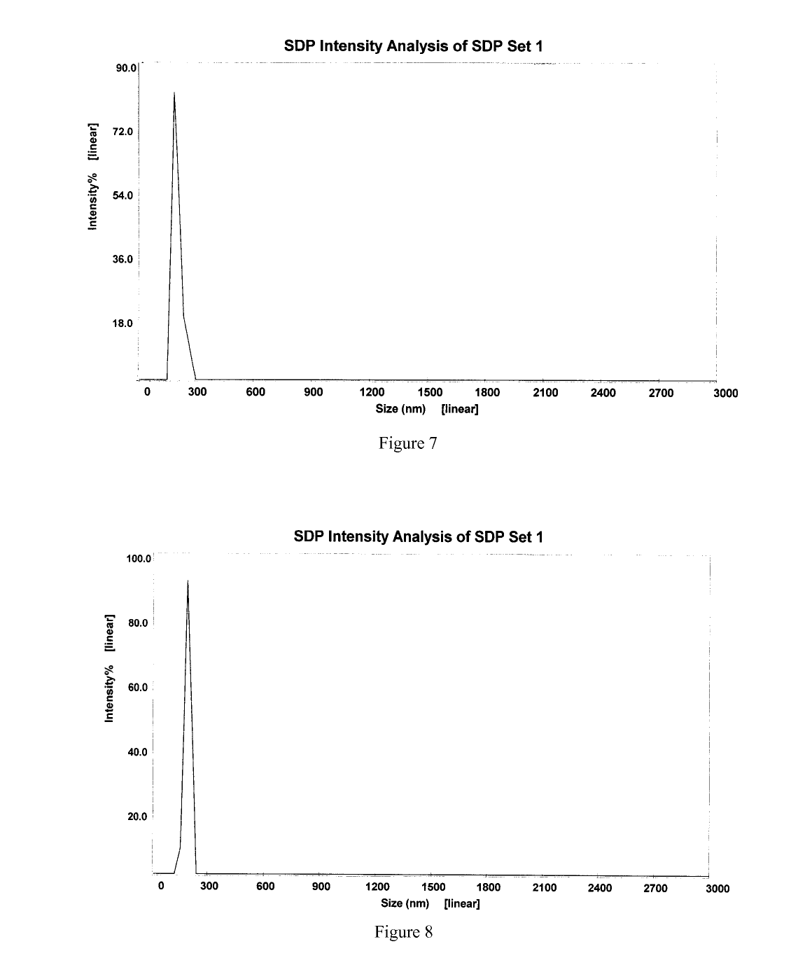

The IR, ¹HNMR, and MS (ESI) spectrograms of the colorless liquid are shown in FIGS. 1-3. The corresponding data is as follows:
IR (cm⁻¹)2968, 2862, 1735, 1516, 1450, 1370, 1118, 950, 760
¹H NMR (300 MHz, CDCl₃) δ(ppm) 0.89 (d, J=6.6 Hz, 6H), 1.41 (d, J=5.4 Hz, J=22.2 Hz, 3H), 1.48 (d, J=7.2, 3H), 1.84 (m, 1H), 2.01 (d, J=31.5 Hz, 2H), 2.44 (d, J=7.2, 2H), 3.68 (m, 1H), 6.85 (m, 1H), 7.09 (m, 2H), 7.18 (m, 2H)
MS (ESI): m/z 608 [2M+Na], 315 [M+Na]

### EXAMPLE 2

Add 103 g (0.5 mol) ibuprofen and 100 g potassium bicarbonate into a 2,500 ml three-neck flask, add 1,000 ml acetone while agitating, add 134 g (0.8 mol) 1-ethyl bromoacetate in droplets at room temperature, and maintain the reaction for 3 h while agitating at 40° C.; then, add 2,000 ml ethyl acetate to dilute the solution, and transfer the reaction liquid into a separatory funnel; wash with 3 wt. % sodium carbonate solution (2×800 ml), and separate to obtain the organic layer; dry with anhydrous sodium sulfate, filter off the drying agent, add active carbon to carry out decolorization with reflux for 20 min., filter off the active carbon, condense the filtrate at normal pressure till no liquid can be distilled off; distil the residue at reduced pressure, and collect 164˜166° C./2 mmHg distillate to obtain 130 g colorless liquid; verified with IR, ¹HNMR, and MS (ESI) spectrograms, the colorless liquid is the target product ibuprofen-1-acetoxy ethyl ester; in relation to the raw material, the yield ratio of ibuprofen is 89%.

### EXAMPLE 3

Add 2,060 g (10 mol) ibuprofen and 240 g potassium bicarbonate into a 5000 ml three-neck flask, add 1000 ml acetone while agitating, add 2,345 g (14 mol) 1-ethyl bromoacetate in droplets at room temperature, and maintain the reaction for 3 h while agitating at 25° C.; then, add 1000 ml ethyl acetate to dilute the solution, and transfer the reaction liquid into a separatory funnel; wash with 3 wt. % sodium carbonate solution (2×5,000 ml), and separate to obtain the organic layer; dry with anhydrous sodium sulfate, filter off the drying agent, add active carbon to carry out decolorization with reflux for 20 min., filter off the active carbon, condense the filtrate at normal pressure till no liquid can be distilled off; distil the residue at reduced pressure, and collect 164˜166° C./2 mmHg distillate to obtain 2,642 g colorless liquid; verified with IR, ¹HNMR, and MS (ESI) spectrograms, the colorless liquid is the target product ibuprofen-1-acetoxy ethyl ester; in relation to the raw material, the yield ratio of ibuprofen is 90.5%.

### EXAMPLE 4

Add 1.03 g (0.005 mol) (R)-(−)-ibuprofen and 0.8 g potassium bicarbonate into a 250 ml three-neck flask, add 15 ml acetone while agitating, add 1.34 g (0.008 mol) 1-ethyl bromoacetate in droplets at room temperature, and maintain the reaction for 3 h while agitating at 25° C.; then, add 20 ml ethyl acetate to dilute the solution, and transfer the reaction liquid into a separatory funnel; wash with 3 wt. % sodium carbonate solution (2×10 ml), and separate to obtain the organic layer; dry with anhydrous sodium sulfate, filter off the drying agent, add active carbon to carry out decolorization with reflux for 20 min., filter off the active carbon, condense the filtrate at normal pressure till no liquid can be distilled off; distil the residue at reduced pressure, and collect 164˜166° C./2 mmHg distillate to obtain 1.34 g colorless liquid; verified with IR, ¹HNMR, and MS (ESI) spectrograms, the colorless liquid is the target product (R)-(−)-ibuprofen-1-acetoxy ethyl ester; in relation to the raw material, the yield ratio of (R)-(−)-ibuprofen is 91.4%, [α]_{D}²⁰=−34.5(c0.03 CH₃OH).

### EXAMPLE 5

Add 20.6 g (0.1 mol) (S)-(+)-ibuprofen and 24 g potassium bicarbonate into a 250 ml three-neck flask, add 100 ml acetone while agitating, add 25.12 g (0.15 mol) 1-ethyl bromoacetate in droplets at room temperature, and maintain the reaction for 3 h while agitating at 25° C.; then, add 100 ml ethyl acetate to dilute the solution, and transfer the reaction liquid into a separatory funnel; wash with 3 wt. % sodium carbonate solution (2×50 ml), and separate to obtain the organic layer; dry with anhydrous sodium sulfate, filter off the drying agent, add active carbon to carry out decolorization with reflux for 20 min., filter off the active carbon, condense the filtrate at normal pressure till no liquid can be distilled off; distil the residue at reduced pressure, and collect 163˜164° C./2 mmHg distillate to obtain 26.72 g colorless liquid; verified with IR, ¹HNMR, and MS (ESI) spectrograms, the colorless liquid is the target product (S)-(+)-ibuprofen-1-acetoxy ethyl ester; in relation to the raw material, the yield ratio of (S)-(+)-ibuprofen is 91.5%, [α]_{D}²⁰=34.5(c0.03 CH₃OH).

### EXAMPLE 6

Add 10.3 g (0.05 mol) ibuprofen and 8 g potassium bicarbonate into a 250 ml three-neck flask, add 110 ml acetone while agitating, add 12.3 g (0.08 mol) 1-ethyl chloroacetate in droplets at room temperature, and maintain the reaction for 5 h while agitating at 25° C.; then, add 200 ml ethyl acetate to dilute the solution, and transfer the reaction liquid into a separatory funnel; wash with 3 wt. % sodium carbonate solution (2×100 ml), and separate to obtain the organic layer; dry with anhydrous sodium sulfate, filter off the drying, add active carbon to carry out decolorization with reflux for 20 min., filter off the active carbon, condense the filtrate at normal pressure till no liquid can be distilled off; distil the residue at reduced pressure, and collect 164˜166° C./2 mmHg distillate to obtain 11.2 g colorless liquid; verified with IR, ¹HNMR, and MS (ESI) spectrograms, the colorless liquid is the target product ibuprofen-1-acetoxy ethyl ester; in relation to the raw material, the yield ratio of ibuprofen is 75.3%.

### EXAMPLE 7

Add 103 g (0.5 mol) ibuprofen and 100 g potassium bicarbonate into a 2,500 ml three-neck flask, add 1,000 ml acetone while agitating, add 123 g (0.8 mol) 1-ethyl chloroacetate in droplets at room temperature, and maintain the reaction for 5 h while agitating at 25° C.; then, add 2,000 ml ethyl acetate to dilute the solution, and transfer the reaction liquid into a separatory funnel; wash with 3 wt. % sodium carbonate solution (2×800 ml), and separate to obtain the organic layer; dry with anhydrous sodium sulfate, filter off the drying agent, add active carbon to carry out decolorization with reflux for 20 min., filter off the active carbon, condense the filtrate at normal pressure till no liquid can be distilled off; distil the residue at reduced pressure, and collect 164˜166° C./2 mmHg distillate to obtain 117 g colorless liquid; verified with IR, ¹HNMR, and MS (ESI) spectrograms, the colorless liquid is the target product ibuprofen-1-acetoxy ethyl ester; in relation to the raw material, the yield ratio of ibuprofen is 80.1%.

### EXAMPLE 8

Add 2,060 g (10 mol) ibuprofen and 240 g potassium bicarbonate into a 5000 ml three-neck flask, add 1000 ml acetone while agitating, add 1,845 g (15 mol) ethyl ester of chloroacetic acid in droplets at room temperature, and maintain the reaction for 5 h while agitating at 25° C.; then, add 1000 ml ethyl acetate to dilute the solution, and transfer the reaction liquid into a separatory funnel; wash with 3 wt. % sodium carbonate solution (2×5,000 ml), and separate to obtain the organic layer; dry with anhydrous sodium sulfate, filter off the drying agent, add active carbon to carry out decolorization with reflux for 20 min., filter off the active carbon, condense the filtrate at normal pressure till no liquid can be distilled off; distil the residue at reduced pressure, and collect 164˜166° C./2 mmHg distillate to obtain 2,371 g colorless liquid; verified with IR, ¹HNMR, and MS (ESI) spectrograms, the colorless liquid is the target product ibuprofen-1-acetoxy ethyl ester; in relation to the raw material, the yield ratio of ibuprofen is 81.2%.

### EXAMPLE 9

Add 10.3 g (0.05 mol) ibuprofen and 6 g potassium bicarbonate into a 250 ml three-neck flask, add 110 ml acetone while agitating, add 11.7 g (0.08 mol) ethylene diacetate in droplets at room temperature, and maintain the reaction for 10 h while agitating at 10° C.; then, add 200 ml ethyl acetate to dilute the solution, and transfer the reaction liquid into a separatory funnel; wash with 3 wt. % sodium carbonate solution (2×100 ml), and separate to obtain the organic layer; dry with anhydrous sodium sulfate, filter off the dryer, add active carbon to carry out decolorization with reflux for 20 min., filter off the active carbon, condense the filtrate at normal pressure till no liquid can be distilled off; distil the residue at reduced pressure, and collect 164˜166° C./2 mmHg distillate to obtain 10.5 g colorless liquid; verified with IR, ¹HNMR, and MS (ESI) spectrograms, the colorless liquid is the target product ibuprofen-1-acetoxy ethyl ester; in relation to the raw material, the yield ratio of ibuprofen is 71.9%.

### EXAMPLE 10

Add 103 g (0.5 mol) ibuprofen and 80 g potassium bicarbonate into a 250 ml three-neck flask, add 110 ml acetone while agitating, add 146 g (1 mol) ethylene diacetate in droplets at room temperature, and maintain the reaction for 10 h while agitating at 25° C.; then, add 2,000 ml ethyl acetate to dilute the solution, and transfer the reaction liquid into a separatory funnel; wash with 3 wt. % sodium carbonate solution (2×800 ml), and separate to obtain the organic layer; dry with anhydrous sodium sulfate, filter off the dryer, add active carbon to carry out decolorization with reflux for 20 min., filter off the active carbon, condense the filtrate at normal pressure till no liquid can be distilled off; distil the residue at reduced pressure, and collect 164˜166° C./2 mmHg distillate to obtain 106 g colorless liquid; verified with IR, ¹HNMR, and MS (ESI) spectrograms, the colorless liquid is the target product ibuprofen-1-acetoxy ethyl ester; in relation to the raw material, the yield ratio of ibuprofen is 72.6%.

### EXAMPLE 11

Add 2,060 g (10 mol) ibuprofen and 200 g potassium bicarbonate into a 5000 ml three-neck flask, add 1000 ml acetone while agitating, add 2,044 g (14 mol) ethylene diacetate in droplets at room temperature, and maintain the reaction for 10 h while agitating at 25° C.; then, add 1000 ml ethyl acetate to dilute the solution, and transfer the reaction liquid into a separatory funnel; wash with 3 wt. % sodium carbonate solution (2×5,000 ml), and separate to obtain the organic layer; dry with anhydrous sodium sulfate, filter off the dryer, add active carbon to carry out decolorization with reflux for 20 min., filter off the active carbon, condense the filtrate at normal pressure till no liquid can be distilled off; distil the residue at reduced pressure, and collect 178˜180° C./3 mmHg distillate to obtain 2,180 g colorless liquid; verified with IR, ¹HNMR, and MS (ESI) spectrograms, the colorless liquid is the target product ibuprofen-1-acetoxy ethyl ester; in relation to the raw material, the yield ratio of ibuprofen is 74.7%.

Examples 12-21 are formulation examples in the present invention.

### EXAMPLE 12

Take 100 g ibuprofen-1-acetoxy ethyl ester prepared in Example 1, 12 g refined egg yolk lecithin, 100 g refined soybean oil, 22 g refined glycerin, 0.3 g refined oleic acid, and sodium hydrogen phosphate in appropriate amount. Mix the ibuprofen-1-acetoxy ethyl ester, refined egg yolk lecithin, refined soybean oil, and refined oleic acid under nitrogen protection, heat up to 75˜80° C. in water bath and agitate to homogeneous state, to obtain ibuprofen-1-acetoxy ethyl ester mixture. Take approx. 766 ml 70˜75° C. water for injection, adjust the pH of the water to 6.5˜6.8 with sodium hydrogen phosphate, add refined glycerin, control a FA25 high-shear dispersion emulsifying machine produced by Shanghai FLUKO Fluid Machine Manufacturing Co., Ltd. to rotate in the water for injection at a high speed to dissolve the glycerin completely; add the ibuprofen-1-acetoxy ethyl ester mixture into the water for injection slowly under nitrogen protection, and keep high-speed shearing for 10˜15 min., to produce mixed emulsion in approx. 1,000 ml total volume; treat the mixed emulsion by high-pressure homogenization for several times in a NS1001H high-pressure homogenizer produced by GEA Niro (Italy), to produce an emulsion formulation with average particle size within 160˜190 nm range; fill the emulsion formulation into 5 ml ampoule bottles to make each ampoule bottle contain 400 mg ibuprofen-1-acetoxy ethyl ester; sterilize for 8min. in water bath at 121° C. under the condition of 121° C. temperature and 8≦F₀<12.

### EXAMPLE 13

Take 200 g ibuprofen-1-acetoxy ethyl ester prepared in Example 2, 12 g refined egg yolk lecithin, 50 g refined soybean oil, 50 g refined mid-chain oil (mid-chain triglyceride), 22 g refined glycerin, 0.3 g refined oleic acid, and sodium hydrogen phosphate in appropriate amount. Mix the ibuprofen-1-acetoxy ethyl ester, refined egg yolk lecithin, refined soybean oil, refined mid-chain oil, and refined oleic acid under nitrogen protection, heat up to 75˜80° C. in water bath and agitate to homogeneous state, to obtain ibuprofen-1-acetoxy ethyl ester mixture. Take approx. 666 ml 70˜75° C. water for injection, adjust the pH of the water to 6.5˜6.8 with sodium hydrogen phosphate, add refined glycerin, control a FA25 high-shear dispersion emulsifying machine produced by Shanghai FLUKO Fluid Machine Manufacturing Co., Ltd. to rotate in the water for injection at a high speed to dissolve the glycerin completely; add the ibuprofen-1-acetoxy ethyl ester mixture into the water for injection slowly under nitrogen protection, and keep high-speed shearing for 10˜15 min., to produce mixed emulsion in approx. 1,000 ml total volume; treat the mixed emulsion by high-pressure homogenization for several times in a NS1001H high-pressure homogenizer produced by GEA Niro (Italy), to produce an emulsion formulation with average particle size within 160˜190 nm range; fill the emulsion formulation into 5 ml ampoule bottles to make each ampoule bottle contain 800 mg ibuprofen-1-acetoxy ethyl ester; sterilize for 15 min. in water bath at 121° C. under the condition of 121° C. temperature and F₀>12.

### EXAMPLE 14

Take 100 g (S)-(+)-ibuprofen-1-acetoxy ethyl ester prepared in Example 5, 12 g refined egg yolk lecithin, 50 g refined soybean oil, 50 g refined mid-chain oil (mid-chain triglyceride), 22 g refined glycerin, 0.3 g refined oleic acid, and sodium hydrogen phosphate in appropriate amount. Mix the (S)-(+)-ibuprofen-1-acetoxy ethyl ester, refined egg yolk lecithin, refined soybean oil, refined mid-chain oil, and refined oleic acid under nitrogen protection in a shaded environment, heat up to 75˜80° C. in water bath and agitate to homogeneous state, to obtain (S)-(+)-ibuprofen-1-acetoxy ethyl ester mixture. Take approx. 766 ml 70˜75° C. water for injection, adjust the pH of the water to 6.5˜6.8 with sodium hydrogen phosphate, add refined glycerin, control a FA25 high-shear dispersion emulsifying machine produced by Shanghai FLUKO Fluid Machine Manufacturing Co., Ltd. to rotate in the water for injection at a high speed to dissolve the glycerin completely; add the (S)-(+)-ibuprofen-1-acetoxy ethyl ester mixture into the water for injection slowly under nitrogen protection, and keep high-speed shearing for 10˜15 min., to produce mixed emulsion in approx. 1,000 ml total volume; treat the mixed emulsion by high-pressure homogenization for several times in a NS1001H high-pressure homogenizer produced by GEA Niro (Italy), to produce an emulsion formulation with average particle size within 160˜190 nm range; fill the emulsion formulation into 5 ml brown ampoule bottles to make each ampoule bottle contain 400 mg (S)-(+)-ibuprofen-1-acetoxy ethyl ester; sterilize in water bath under the condition of 121° C. temperature and F₀>8; sterilize in water bath at 126° C. for 5 min. under the condition of 126° C. temperature and F₀>12.

### EXAMPLE 15

Take 100 g (R)-(−)-ibuprofen-1-acetoxy ethyl ester prepared in Example 4, 12 g refined egg yolk lecithin, 50 g refined soybean oil, 50 g refined mid-chain oil (mid-chain triglyceride), 22 g refined glycerin, 0.3 g refined oleic acid, and sodium hydrogen phosphate in appropriate amount. Mix the (R)-(+)-ibuprofen-1-acetoxy ethyl ester, refined egg yolk lecithin, refined soybean oil, refined mid-chain oil, and refined oleic acid under nitrogen protection in a shaded environment, heat up to 75˜80° C. in water bath and agitate to homogeneous state, to obtain (R)-(+)-ibuprofen-1-acetoxy ethyl ester mixture. Take approx. 766 ml 70˜75° C. water for injection, adjust the pH of the water to 6.5˜6.8 with sodium hydrogen phosphate, add refined glycerin, control a FA25 high-shear dispersion emulsifying machine produced by Shanghai FLUKO Fluid Machine Manufacturing Co., Ltd. to rotate in the water for injection at a high speed to dissolve the glycerin completely; add the (R)-(+)-ibuprofen-1-acetoxy ethyl ester mixture into the water for injection slowly under nitrogen protection, and keep high-speed shearing for 10˜15 min., to produce mixed emulsion in approx. 1,000 ml total volume; treat the mixed emulsion by high-pressure homogenization for several times in a NS1001H high-pressure homogenizer produced by GEA Niro (Italy), to produce an emulsion formulation with average particle size within 160˜190 nm range; fill the emulsion formulation into 5 ml brown ampoule bottles to make each ampoule bottle contain 400 mg (R)-(−)-ibuprofen-1-acetoxy ethyl ester; sterilize in water bath at 115° C. for 30 min. under the condition of 115° C. temperature and 8≦F₀<12.

### EXAMPLE 16

Take 10 g ibuprofen-1-acetoxy ethyl ester prepared in Example 3, 40 g refined soya bean lecithin with lecithin content not lower than 75%, 10 g refined cholesterol, 1 g refined oelic acid, and 100 ml medicinal ethanol. Agitate the ibuprofen-1-acetoxy ethyl ester, soya bean lecithin, cholesterol, and oleic acid in water bath at 65˜70° C. temperature under nitrogen protection, with the dissolution aiding function of medicinal ethanol, to obtain ibuprofen-1-acetoxy ethyl ester mixture. Prepare approx. 940 ml pH6.8 disodium hydrogen phosphate-sodium dihydrogen phosphate buffer solution, heat up the buffer solution in water bath to 70˜75° C., control a FA25 high-shear dispersion emulsifying machine produced by Shanghai FLUKO Fluid Machine Manufacturing Co., Ltd. to rotate in the solution at a high speed; add the ibuprofen-1-acetoxy ethyl ester mixture into the solution slowly under nitrogen protection, keep high-speed shearing for 10˜15 min., and decrease the pressure to remove the ethanol and produce a mixed emulsion; treat the mixed emulsion by high-pressure homogenization for several times in a NS1001H high-pressure homogenizer produced by GEA Niro (Italy), to produce a liposome translucent emulsion with average particle size within 120˜160 nm range; fill the emulsion into 5 ml ampoule bottles to make each ampoule bottle contain 40 mg ibuprofen-1-acetoxy ethyl ester; sterilize for 45 min. in water bath at 100° C.

### EXAMPLE 17

Take 10 g (R)-(−)-ibuprofen-1-acetoxy ethyl ester prepared in Example 4, 40 g refined soya bean lecithin with lecithin content not lower than 75%, 10 g refined cholesterol, 1 g refined oelic acid, and 100 ml medicinal ethanol. Agitate the (R)-(+)-ibuprofen-1-acetoxy ethyl ester, soya bean lecithin, cholesterol, and oleic acid in water bath at 65˜70° C. temperature under nitrogen protection, with the dissolution aiding function of medicinal ethanol, to obtain (R)-(+)-ibuprofen-1-acetoxy ethyl ester mixture. Prepare 940 ml pH6.8 disodium hydrogen phosphate-sodium dihydrogen phosphate buffer solution, heat up the buffer solution in water bath to 70˜75° C., control a FA25 high-shear dispersion emulsifying machine produced by Shanghai FLUKO Fluid Machine Manufacturing Co., Ltd. to rotate in the solution at a high speed; add the ibuprofen-1-acetoxy ethyl ester mixture into the solution slowly under nitrogen protection, keep high-speed shearing for 10˜15 min., and decrease the pressure to remove the ethanol, add water for injection to approx. 1,000 ml total volume to produce a mixed emulsion; treat the mixed emulsion by high-pressure homogenization for several times in a NS1001H high-pressure homogenizer produced by GEA Niro (Italy), to produce a liposome translucent emulsion with average particle size within 120˜160 nm range; fill the emulsion into 5 ml ampoule bottles to make each ampoule bottle contain 40 mg (R)-(−)-ibuprofen-1-acetoxy ethyl ester; sterilize for 45 min. in water bath at 110° C. under the conditions of 110° C. temperature and 8≦F₀<12.

### EXAMPLE 18

Take 10 g (S)-(+)-ibuprofen-1-acetoxy ethyl ester prepared in Example 5, 40 g refined soya bean lecithin with lecithin content not lower than 75%, 10 g refined cholesterol, 1 g refined oelic acid, and 100 ml medicinal ethanol. Agitate the (S)-(+)-ibuprofen-1-acetoxy ethyl ester, soya bean lecithin, cholesterol, and oleic acid in water bath at 65˜70° C. temperature under nitrogen protection, with the dissolution aiding function of medicinal ethanol, to obtain (S)-(+)-ibuprofen-1-acetoxy ethyl ester mixture. Prepare 940 ml pH6.8 disodium hydrogen phosphate-sodium dihydrogen phosphate buffer solution, heat up the buffer solution in water bath to 70˜75° C., control a FA25 high-shear dispersion emulsifying machine produced by Shanghai FLUKO Fluid Machine Manufacturing Co., Ltd. to rotate in the solution at a high speed; add the ibuprofen-1-acetoxy ethyl ester mixture into the solution slowly under nitrogen protection, keep high-speed shearing for 10˜15 min., and decrease the pressure to remove the ethanol, add water for injection to approx. 1,000 ml total volume to produce a mixed emulsion; treat the mixed emulsion by high-pressure homogenization for several times in a NS1001H high-pressure homogenizer produced by GEA Niro (Italy), to produce a liposome translucent emulsion with average particle size within 120˜160 nm range; fill the emulsion into 5 ml ampoule bottles to make each ampoule bottle contain 40 mg (S)-(+)-ibuprofen-1-acetoxy ethyl ester; sterilize for 15 min. in water bath at 121° C. under the conditions of 121° C. temperature and F₀>12.

### EXAMPLE 19

Take 100 g ibuprofen-1-acetoxy ethyl ester prepared in Example 6, 15 g refined lecithin, 100 g refined soybean oil, 0.5 g refined sodium oleate, 2 g lactose, and 22 g refined glycerin. Agitate the ibuprofen-1-acetoxy ethyl ester, refined lecithin, refined soybean oil, and refined oleic acid in water bath at 65˜70° C. temperature under nitrogen protection, to obtain ibuprofen-1-acetoxy ethyl ester mixture. Take approx. 780 ml 70˜75° C. water for injection, adjust pH of the water with sodium citrate to 6.5˜6.8 to produce a buffer solution, dissolve the lactose and refined glycerin into the water, control a FA25 high-shear dispersion emulsifying machine produced by Shanghai FLUKO Fluid Machine Manufacturing Co., Ltd. to rotate in the water for injection at a high speed; add the ibuprofen-1-acetoxy ethyl ester mixture into the water for injection slowly under nitrogen protection, and keep high-speed shearing for 10˜15min., to produce a mixed emulsion; treat the mixed emulsion by high-pressure homogenization for several times in a NS1001H high-pressure homogenizer produced by GEA Niro (Italy), to produce an emulsion with average particle size within 160˜190 nm range; fill the emulsion into 5 ml vials to make each vial contain 400 mg ibuprofen-1-acetoxy ethyl ester; freeze the emulsion in a freezing dryer to −30˜60° C. so that the emulsion solidifies; then, heat up by stages to 0˜40° C. in a high vacuum environment, whiling controlling the freeze-drying curve, to obtain dried emulsion of ibuprofen-1-acetoxy ethyl ester finally.

### EXAMPLE 20

Take 100 g (S)-(+)-ibuprofen-1-acetoxy ethyl ester prepared in Example 5, 15 g refined lecithin, 100 g refined soybean oil, 0.5 g refined oelic acid, 2 g lactose, 22 g refined glycerin, and sodium citrate in appropriate amount. Agitate the (S)-(+)-ibuprofen-1-acetoxy ethyl ester, refined lecithin, refined soybean oil, and refined oleic acid in water bath at 65˜70° C. temperature under nitrogen protection, to obtain (S)-(+)-ibuprofen-1-acetoxy ethyl ester mixture. Take approx. 780 ml 70˜75° C. water for injection, adjust the pH of the water to 6.5˜6.8 with sodium citrate to produce a buffer solution, dissolve lactose and refined glycerin in the water, control a FA25 high-shear dispersion emulsifying machine produced by Shanghai FLUKO Fluid Machine Manufacturing Co., Ltd. to rotate in the water for injection at a high speed; add the (S)-(+)-ibuprofen-1-acetoxy ethyl ester mixture into the water for injection slowly under nitrogen protection, and keep high-speed shearing for 10˜15 min., to produce a mixed emulsion; treat the mixed emulsion by high-pressure homogenization for several times in a NS1001H high-pressure homogenizer produced by GEA Niro (Italy), to produce an emulsion with average particle size within 160˜180 nm range; fill the emulsion formulation into 5 ml vials to make each vial contain 400 mg (S)-(+)-ibuprofen-1-acetoxy ethyl ester; freeze the emulsion in a freezing dryer to −30˜60° C. so that the emulsion solidifies; then, heat up the emulsion by stages to 0˜40° C. in a high vacuum environment, while controlling the freeze-drying curve; to obtain dried emulsion of (S)-(+)-ibuprofen-1-acetoxy ethyl ester finally.

### EXAMPLE 21

Take 100 g (R)-(−)-ibuprofen-1-acetoxy ethyl ester prepared in Example 4, 15 g refined lecithin, 100 g refined soybean oil, 0.5 g refined oelic acid, 2 g lactose, 22 g refined glycerin, and sodium citrate in appropriate amount. Agitate the (R)-(+)-ibuprofen-1-acetoxy ethyl ester, refined lecithin, refined soybean oil, and refined oleic acid in water bath at 65˜70° C. temperature under nitrogen protection, to obtain (R)-(−)-ibuprofen-1-acetoxy ethyl ester mixture. Take approx. 780 ml 70˜75° C. water for injection, adjust the pH of the water to 6.5˜6.8 with sodium citrate to produce a buffer solution, dissolve lactose and refined glycerin in the water, control a FA25 high-shear dispersion emulsifying machine produced by Shanghai FLUKO Fluid Machine Manufacturing Co., Ltd. to rotate in the water for injection at a high speed; add the (R)-(−)-ibuprofen-1-acetoxy ethyl ester mixture into the water for injection slowly under nitrogen protection, and keep high-speed shearing for 10˜15 min., to produce a mixed emulsion; treat the mixed emulsion by high-pressure homogenization for several times in a NS1001H high-pressure homogenizer produced by GEA Niro (Italy), to produce an emulsion formulation with average particle size within 160˜180 nm range; fill the emulsion formulation into 5 ml vials to make each vial contain 400mg (R)-(+)-ibuprofen-1-acetoxy ethyl ester; freeze the emulsion in a freezing dryer to −30° C.˜60° C. so that the emulsion solidifies; then, heat up the emulsion by stages to 0˜40° C. in a high vacuum environment while controlling the freeze-drying curve, to obtain dried emulsion of (R)-(+)-ibuprofen-1-acetoxy ethyl ester finally.

Examples 22-23 are examples provided to demonstrate the drug effect of the present invention.

### EXAMPLE 22

### (I) Sample Selection

Take 12 test Beagle dogs raised by ourselves (8-12 kg body weight, a half of the dogs are male ones, and the other half of the dogs are female ones), divide them into test group 1, test group 2, comparative group 1 and comparative group 2 in random, with 3 dogs in each group. Control them in empty stomach state within 12 h before dosing, but don't restrict drinking in that period.

### (II) Prepare a Standard Drug-Time Curve

On the test day, take 100 μl standard ibuprofen solution and add it into a centrifuge tube (EP tube). Select one Beagle dog in the comparative group 2 in random, take 100 μl blank blood sample from the dog, and add the blank blood sample into the EP tube, and then add 100 μl internal standard felbinac and 300 μl acetonitrile into the EP tube. Then, use a turbine mixer, which is well-known in the art; load the EP tube into the turbine mixer and rotate for 1 min, to mix the solution in the tube to homogeneous state. Next, treat by centrifugation for 5 min. at 15,000 rpm speed in a centrifugal machine well known in the art, hold for 10 min., and suck up the supernatant serum in the EP tube with a transfer pipette well known in the art and transfer the serum into a different test tube. Analyze by liquid chromatography-mass spectrometry/mass spectrometry (LC-MS/MS), and prepare a standard drug-time curve.

### (III) Determine the Dosage

Convert the dosage of mid-chain/long-chain lipid emulsion in the ibuprofen-1-acetoxy ethyl ester prepared in Example 13 (the content of mid-chain/long-chain lipid emulsion in the ibuprofen-1-acetoxy ethyl ester is 100 mg/ml, equivalent to 70 mg/ml ibuprofen content) for Beagle dogs, on the basis of 400 mg ibuprofen/kg body weight for human beings. The conversion result is: the dosage for Beagle dogs is 12.5 mg ibuprofen/kg body weight.

### (IV) Prepare Blood Samples

Complete intravenous injection for the test group 1 within 0.17 h and oral administration for the test group 2, with the dosage determined in step (III). Take 1 ml blood from the vena saphena parva in a rear leg for the test groups 1 and 2 at the times shown in the following table 1 and table 2 respectively, and load the blood into heparin tubes that contain cholinesterase inhibitor respectively, to obtain blood samples.

### (V) Test Blood Samples

Take 100 μl blood sample obtained in step (IV), add 100 μl internal standard felbinac, and add 400 μl acetonitrile respectively; rotate the blood sample for 1 min. in a turbine mixer to mix the solution in the tube to homogeneous state; then, treat the sample by centrifugation for 5 min. at 15,000 rpm speed in a centrifugal machine, place for 10 min., take the supernatant serum, and analyze by LC-MS/MS.

The pharmacokinetic parameters of mid-chain/long-chain lipid emulsion in the ibuprofen-1-acetoxy ethyl ester during intravenous injection for test group 1 are shown in the following table 1:

TABLE 1
Pharmacokinetic Parameters of Mid-Chain/Long-Chain Lipid Emulsion in the
Ibuprofen-1-Acetoxy Ethyl Ester during Intravenous Injection for Test Group 1

| | | | | | Standard |
|---|---|---|---|---|---|
| | Plasma | Plasma | Plasma | | deviation of |
| Blood | Concentration | Concentration | Concentration | Mean Plasma | Plasma |
| sampling time | of Beagle dog | of Beagle dog | of Beagle dog | Concentration | Concentration |
| (h) | 1# (ng/ml) | 2# (ng/ml) | 3# (ng/ml) | (ng/ml) | (±SD) |
| 0 | 0 | 0 | 0 | 0 | 0 |
| 0.033 | 76800 | 52700 | 75500 | 68333 | 13554 |
| 0.083 | 55600 | — | 60700 | 58150 | 3606 |
| 0.17 | 55500 | 46700 | 55900 | 52700 | 5200 |
| 0.33 | 49400 | 39100 | 51400 | 46633 | 6600 |
| 0.5 | 44300 | 36200 | 44100 | 41533 | 4620 |
| 0.75 | 34500 | 39300 | 34600 | 36133 | 2743 |
| 1 | 31100 | 30800 | 32600 | 31500 | 964 |
| 2 | 26500 | 17000 | 25200 | 22900 | 5151 |
| 3 | 20500 | 10500 | 17000 | 16000 | 5074 |
| 5 | 14700 | 4700 | 9240 | 9547 | 5007 |
| 7 | 9550 | 2670 | 5810 | 6010 | 3444 |
| 12 | 2470 | 664 | 1370 | 1501 | 910 |
| 24 | 600 | 167 | 184 | 317 | 245 |
| AUC₀₋ₜ | 204039.2 | 112671 | 163223.2 | 159978 | 45770 |
| (ng/mL*h) | | | | | |
| (t = 24 h) | | | | | |
| Cₘₐₓ(ng/mL) | 76800 | 52700 | 75500 | 68333 | 13554.458 |
| t_{1/2} ( h) | 4.17 | 4.06 | 3.38 | 3.87 | 0.43 |

\textcircled{1} No blood sample is obtained.

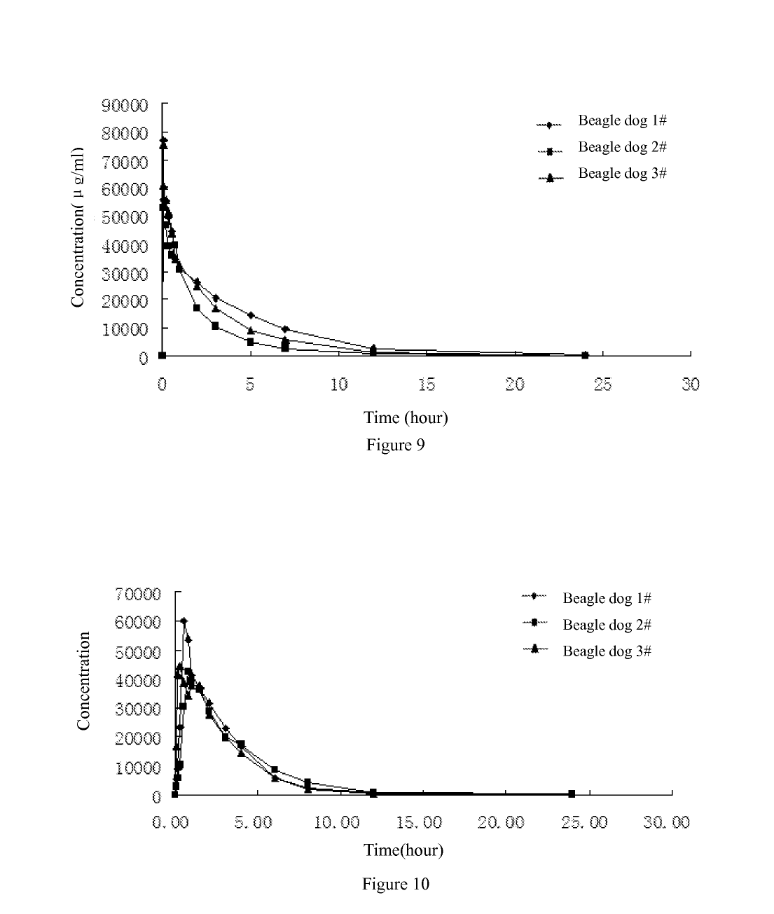

The drug-time curves of mid-chain/long-chain lipid emulsion in ibuprofen-1-acetoxy ethyl ester during intravenous injection for test group 1 are shown in FIG. 9. The variation of plasma concentration of Beagle dog 1#, 2#, and 3# according to time can be seen in FIG. 9. The pharmacokinetic parameters of mid-chain/long-chain lipid emulsion in the ibuprofen-1-acetoxy ethyl ester during oral dosing for test group 2 are shown in the following table 2:

TABLE 2
Pharmacokinetic Parameters of Mid-Chain/Long-Chain Lipid Emulsion in the
Ibuprofen-1-Acetoxy Ethyl Ester during Oral Dosing for Test Group 2

| | | | | | Standard |
|---|---|---|---|---|---|
| | Plasma | Plasma | Plasma | | deviation of |
| Blood | Concentration | Concentration | Concentration | Mean Plasma | Plasma |
| sampling time | of Beagle dog | of Beagle dog | of Beagle dog | Concentration | Concentration |
| (h) | 1# (ng/ml) | 2# (ng/ml) | 3# (ng/ml) | (ng/ml) | (±SD) |
| 0.00 | 0 | 0 | 0 | 0 | 0 |
| 0.083 | 5190 | 2990 | 17000 | 8393 | 7534 |
| 0.167 | 8880 | 5590 | 41700 | 18723 | 19966 |
| 0.250 | 23300 | 10100 | 44300 | 25900 | 17248 |
| 0.500 | 60100 | 30400 | 38600 | 43033 | 15338 |
| 0.750 | 53400 | 42400 | 34000 | 43267 | 9729 |
| 1.0 | 41100 | 39000 | 37700 | 39267 | 1716 |
| 1.5 | 36900 | 36200 | 37800 | 36967 | 802 |
| 2.0 | 31800 | 28800 | 27400 | 29333 | 2248 |
| 3.0 | 23200 | 19900 | 19600 | 20900 | 1997 |
| 4.0 | 16900 | 17200 | 14500 | 16200 | 1480 |
| 6.0 | 5570 | 8780 | 5830 | 6727 | 1783 |
| 8.0 | 2380 | 4270 | 2030 | 2893 | 1205 |
| 12.0 | 625 | 990 | 438 | 684 | 281 |
| 24.0 | 157 | 326 | 97.3 | 193 | 119 |
| 48.0 | n.d. | n.d. | n.d. | 0 | 0 |
| AUC₀₋ₜ | 163915 | 160869 | 147200 | 157328 | 8902 |
| (ng/mL*h) | | | | | |
| (t = 24 h) | | | | | |
| Cₘₐₓ(ng/mL) | 60100 | 42400 | 44300 | 48933 | 9717 |
| t_{1/2} ( h) | 3.74 | 4.03 | 3.29 | 3.69 | 0.37 |

\textcircled{1} Not detectable because out-of-limit.

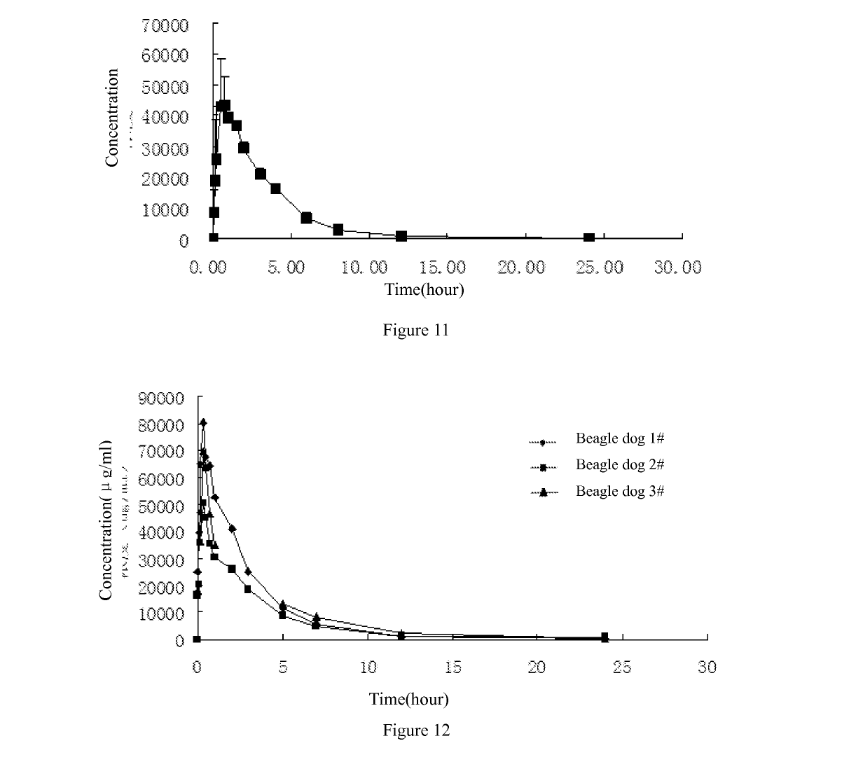

The drug-time curves of mid-chain/long-chain lipid emulsion in the ibuprofen-1-acetoxy ethyl ester in oral dosing for test group 2 are shown in FIG. 10, and the mean drug-time curve is shown in FIG. 11. The variation of plasma concentration of Beagle dog 1#, 2#, and 3# according to time can be seen in FIG. 10. The variation of mean plasma concentration of Beagle dog 1#, 2#, and 3# according to time can be seen in FIG. 11.

It is seen from the above analysis: the AUC₀₋ₜ of mid-chain/long-chain lipid emulsion in the ibuprofen-1-acetoxy ethyl ester is 156,258±8,902 ng/mL*h during oral dosing and 159,978±45,770 ng/mL*h during intravenous injection; compared to intravenous injection, the bioavailability of oral dosing is 97.67%. In addition, the peak value can be attained more quickly during intravenous injection.

### EXAMPLES 23-31

In the examples 23-31, the sample selection, standard drug-time curve preparation, drug dosage determination, blood sample preparation, and blood sample test are carried out with the methods used in Example 22 except that: only intravenous injection is used, and the mid-chain/long-chain lipid emulsion of ibuprofen-1-acetoxy ethyl ester prepared in Example 13 is replaced with the (S)-(+)-ibuprofen-1-acetoxy ethyl ester lipid emulsion prepared in Example 14, (R)-(−)-ibuprofen-1-acetoxy ethyl ester lipid emulsion prepared in Example 15, ibuprofen-1-acetoxy ethyl ester liposome emulsion prepared in Example 16, (S)-(+)-ibuprofen-1-acetoxy ethyl ester liposome emulsion prepared in Example 18, (R)-(−)-ibuprofen-1-acetoxy ethyl ester liposome emulsion prepared in Example 17, ibuprofen-1-acetoxy ethyl ester liposome emulsion prepared in Example 19, (S)-(+)-ibuprofen-1-acetoxy ethyl ester liposome emulsion prepared in Example 20, (R)-(−)-ibuprofen-1-acetoxy ethyl ester liposome emulsion prepared in Example 21, and ibuprofen-1-acetoxy ethyl ester liposome emulsion prepared in Example 12, respectively. The measured pharmacokinetic parameters are:
Example 23: AUC₀₋ₜ is 157±65(μg/mL*h)(t=24 h); Tₘₐₓ is (0.5±0.01) h; Cₘₐₓ is (43.56±7.2)μg·mL⁻¹; T_{1/2} is (3.15±0.1)h.
Example 24: AUC₀₋ₜ is 158.50±30 (μg/mL*h)(t=24 h); Tₘₐₓ is (0.2±0.00)h; Cₘₐₓ is (39.37±7.8)μg·mL⁻¹; T_{1/2} is (2.9±0.1)h.
Example 25: AUC₀₋ₜ is 143.92±55 (μg/mL*h)(t=24 h); Tₘₐₓ is (0.2±0.0)h; Cₘₐₓ is (42.5±7.7)μg·mL⁻¹; T_{1/2} is (3.1±0.1)h.
Example 26: AUC₀₋ₜ is 159.97±45 (μg/mL*h)(t=24 h); Tₘₐₓ is (0.2±0.01)h; Cₘₐₓ is (45.7±7.6)μg·mL⁻¹; T_{1/2} is (3.8±0.1)h.
Example 27: AUC₀₋ₜ is 156.19±40 (μg/mL*h)(t=24 h); Tₘₐₓ is (0.2±0.01)h; Cₘₐₓ is (46.3±7.7)μg·mL⁻¹; T_{1/2} is (2.8±0.2)h.
Example 28: AUC₀₋ₜ is 135.99±57 (μg/mL*h)(t=24 h); Tₘₐₓ is (0.2±0.01)h; Cₘₐₓ is (33.4±7.1)μg·mL⁻¹; T_{1/2} is (3.0±0.1)h.
Example 29: AUC₀₋ₜ is 155.75±35 (μpg/mL*h)(t=24h); Tₘₐₓ is (0.2±0.01)h; Cₘₐₓ is (45.3±6.6)μg·mL⁻¹; T_{1/2} is (3.5±0.1)h.
Example 30: AUC₀₋ₜ is 159.39±55 (μg/mL*h)(t=24 h); Tₘₐₓ is (0.2±0.01)h; Cₘₐₓ is (40.5±8.7)μg·mL⁻¹; T_{1/2} is (2.5±0.2)h.
Example 31: AUC₀₋ₜ is 135.75±45 (μg/mL*h)(t=24 h); Tₘₐₓ is (0.2±0.01)h; Cₘₐₓ is (43.5±8.7)μg·mL⁻¹; T_{1/2} is (3.1±0.2)h.

### COMPARATIVE EXAMPLE 1

Convert the dosage of an ibuprofen injection produced by Cumberland Pharmaceuticals Corporation (USA) (the principal ingredient is ibuprofen) for Beagle dogs, on the basis of 400 mg ibuprofen/kg body weight for human beings. The conversion result is: the dosage for Beagle dogs is 12.5 mg ibuprofen/kg body weight. With reference to the product instructions of the project, dilute every 1.25 ml ibuprofen injection with 30 ml normal saline, and administrate each Beagle dog in the comparative group 1 described in the Example 22 by intravenous infusion within 0.17 h. After administration, take 1 ml blood from the vena saphena parva in a rear leg for the comparative group 1 at the times shown in the following table 3, and load the blood into heparin tubes that contain cholinesterase inhibitor respectively, to obtain blood samples. Test the blood samples with the method used in Example 22, with reference to the standard drug-time curve prepared in Example 22.

The pharmacokinetic parameters during intravenous infusion of the ibuprofen injection for the comparative group are shown in table 3:

TABLE 3
Pharmacokinetic Parameters during Intravenous Infusion of the Ibuprofen Injection

| | Plasma | Plasma | Plasma | | Standard deviation |
|---|---|---|---|---|---|
| Blood | concentration | concentration | concentration | Mean Plasma | of Plasma |
| sampling | of Beagle dog | of Beagle dog | of Beagle dog | concentration | concentration |
| time (h) | 1# (ng/ml) | 2# (ng/ml) | 3# (ng/ml) | (ng/ml) | (±SD) |
| 0 | 0 | 0 | 0 | 0 | 0 |
| 0.033 | 25300 | 16500 | 18200 | 20000 | 4668 |
| 0.083 | 39700 | 20500 | — | 30100 | 13576 |
| 0.117 | 47200 | — | 36100 | 41650 | 7849 |
| 0.17 | 64700 | 35900 | 36800 | 45800 | 16374 |
| 0.33 | 80500 | 50100 | 69700 | 66767 | 15411 |
| 0.5 | 67300 | 44900 | 64000 | 58733 | 12093 |
| 0.75 | 64500 | 35400 | 46600 | 48833 | 14678 |
| 1 | 52600 | 30400 | 34800 | 39267 | 11755 |
| 2 | 41000 | 26200 | — | 33600 | 10465 |
| 3 | 25100 | 18700 | — | 21900 | 4525 |
| 5 | 11500 | 8760 | 13400 | 11220 | 2333 |
| 7 | 5740 | 4860 | 8270 | 6290 | 1770 |
| 12 | 1460 | 1530 | 2420 | 1803 | 535 |
| 24 | 515 | 1040 | 490 | 682 | 311 |
| AUC₀₋ₜ | 225361.7 | 160120.8 | 210660 | 198714 | 34222 |
| (ng/mL*h) | | | | | |
| (t = 24 h) | | | | | |
| Cₘₐₓ(ng/mL) | 80500 | 50100 | 69700 | 66767 | 15411 |
| t_{1/2} ( h) | 4.47 | 6.68 | 4.05 | 4.92 | 6.62 |

\textcircled{1} No blood sample is obtained.

The drug-time curve of the ibuprofen injection in comparative group 1 after intravenous injection is shown in FIG. 12. The variation of plasma concentration of Beagle dog 1#, 2#, and 3# with time can be seen in FIG. 12.

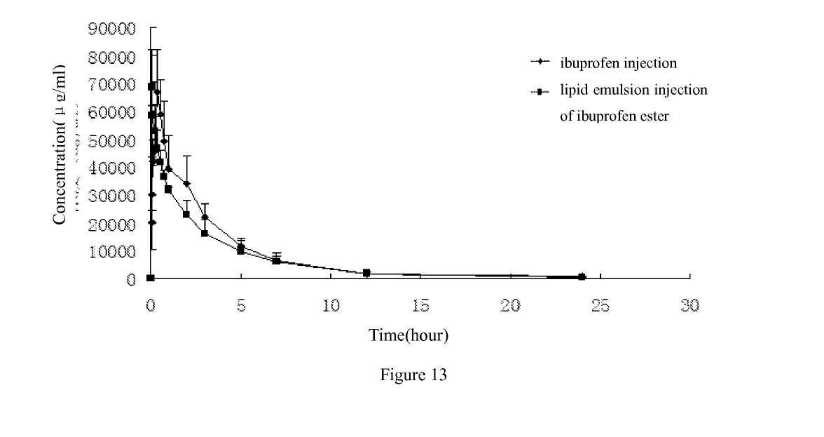

The mean drug-time curve of the ibuprofen injection in comparative group 1 after intravenous injection and the mean drug-time curve of the medium-chain/long-chain lipid emulsion of ibuprofen-1-acetoxy ethyl ester in test group 1 in Example 22 after intravenous injection are shown in FIG. 13. The variation of plasma concentration of Beagle dogs 1#, 2#, and 3# according to time in the comparative group after the ibuprofen injection is used and the variation of plasma concentration of Beagle dogs 1#, 2#, and 3# according to time in the test group after the ibuprofen lipid emulsion is used can be seen in FIG. 13.

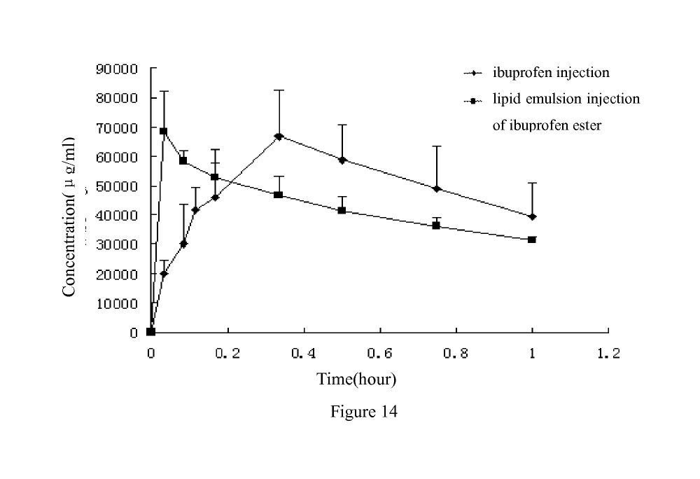

The mean drug-time curve of the ibuprofen injection in comparative group 1 and the mean drug-time curve of the medium-chain/long-chain lipid emulsion of ibuprofen-1-acetoxy ethyl ester in test group 1 in example 22 within 1 h after intravenous injection are shown in FIG. 14. The variation of plasma concentration of Beagle dogs 1#, 2#, and 3# with time in the comparative group within 1 h after the ibuprofen injection is used and the variation of plasma concentration of Beagle dogs 1#, 2#, and 3# according to time in the test group within 1 h after the ibuprofen lipid emulsion is used can be seen in FIG. 14.

Verified by variance test with SPSS software, there is no significant difference (P>0.05) in the pharmacokinetic parameters AUC₀₋ₜ, Cₘₐₓ, t_{1/2} between the comparative group 1 and the test group 1 in Example 22.

It is seen from above comparative study:

Compared to the ibuprofen injection in Comparative Example 1, the ibuprofen ester-based injection formulation prepared in the present invention can reach the expected plasma concentration of ibuprofen within 0.033 h after intravenous injection, possibly because the lipid microspheres of the drug bind with the plasma proteins after intravenous injection and the drug in the lipid microspheres is hydrolyzed quickly by the esterase in the blood into active metabolite ibuprofen. The ibuprofen ester-based injection formulation prepared in the present invention can attain the drug effect of ibuprofen injection while selectively inhibiting COX-2. Compared to the ibuprofen injection in Comparative Example 1, the ibuprofen ester-based oral formulation prepared in the present invention can reach peak blood concentration of ibuprofen within 0.5 h after oral administration, i.e., the time to peak concentration is shorter. The ibuprofen ester-based oral formulation has higher bioavailability and persistent drug action, and is convenient to use.

What is claimed is:

1. An ibuprofen-based compound, having a structure represented by one of structural formula (2), 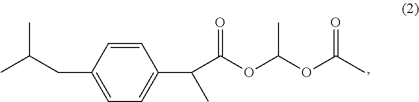 [SMILES: CC(=O)OC(C)OC(=O)C(C)c1ccc(CC(C)C)cc1]
a levorotatory enantiomer of the compound represented by structural formula (2) and having a structure represented by structural formula (3), 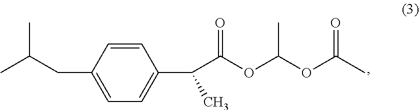 [SMILES: CC(=O)OC(C)OC(=O)[C@H](C)c1ccc(CC(C)C)cc1]
or a dextrorotatory enantiomer of the compound represented by structural formula (2) and having a structure represented by structural formula (4),

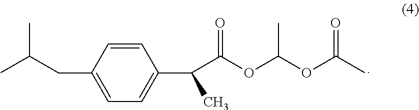 [SMILES: CC(=O)OC(C)OC(=O)[C@@H](C)c1ccc(CC(C)C)cc1]

2-9. (canceled)

10. A method of formulating an NSAID comprising providing the ibuprofen-based compound as set forth in claim 1.

11. A formulation that contains the ibuprofen-based compound as set forth in claim 1, wherein, calculated on the basis of the total weight of the formulation, the content of the ibuprofen-based compound is 1-99 wt. %.

12. The formulation according to claim 11, wherein the formulation is in the form of lipid emulsion injection and includes auxiliary materials that contain an oily matrix phase, lecithin, oleic acid, and glycerin.

13. The formulation according to claim 11, wherein the formulation is in the form of frozen dried emulsion injection and includes auxiliary materials that contain an oily matrix phase, phosphatidylcholine, glycerin, lactose, and oleic acid or sodium oleate.

14. The formulation according to claim 12, wherein the oily matrix phase is one or more selected from the group consisting of long-chain fatty acids and mid-chain fatty acids.

15. The formulation according to claim 11, wherein the formulation is in the form of a liposome injection and includes auxiliary materials that contain phosphatidylcholine, cholesterol, and oleic acid or sodium oleate.

16. The formulation according to claim 13, wherein the oily matrix phase is one or more selected from the group consisting of long-chain fatty acids and mid-chain fatty acids.
